(19) United States
(12) Patent Application Publication
(10) Pub. No.: US 2009/0181087 A1
(43) Pub. Date: Jul. 16, 2009

(54) Use of human cord blood-derived pluripotent cells for the treatment of disease

(75) Inventors: Morey Kraus, Harvard, MA (US); Marc D. Beer, Sudbury, MA (US); Paul T. Clark, Wellesley, MA (US)

Correspondence Address: CLARK & ELBING LLP, 101 FEDERAL STREET, BOSTON, MA, 02110, US

(73) Assignee: Viacell, Inc., Cambridge, MA (US)

(21) Appl. No.: 10/583,684

(22) PCT Filed: Dec. 20, 2004
(86) PCT No.: PCT/US04/42743

§ 371 Date: Nov. 2, 2006

Related U.S. Application Data
(60) Provisional application No. 60/531,577, filed on Dec. 19, 2003.

(51) Int. Cl. A61K 9/00; A61K 35/12; C12Q 1/02; C12N 5/02

(52) U.S. Cl. 424/484; 424/93.7; 435/29; 435/378

(57) ABSTRACT

The present invention features methods of organ tissue regeneration using pluripotent cells derived from umbilical cord blood, compositions of these pluripotent cells, methods for further transforming these cells, and uses for these transformed cells.

### BACKGROUND OF THE INVENTION

The present invention relates to the treatment of disease using pluripotent cells.

A number of types of mammalian pluripotent cells have been characterized. For example, embryonic stem cells, embryonic germ cells, or adult stem cells are known. Caplan et al. (U.S. Pat. No. 5,486,359) describe human mesenchymal stem cells (hMSCs) derived from the bone marrow that serve as progenitors for mesenchymal cell lineages. These hMSCs are identified through the use of monoclonal antibodies that bind to cell surface markers. According to Caplan et al., homogeneous hMSC compositions are obtained by the positive selection of adherent marrow or periosteal cells free of markers associated with either hematopoietic cell or differentiated mesenchymal cells. The isolated mesenchymal cell populations display epitopic characteristics associated with mesenchymal stem cells, have the ability to regenerate in culture without differentiating, and have the ability to differentiate into specific mesenchymal lineages when either induced in vitro or placed in vivo at the site of damaged tissue. The method requires harvesting of marrow or periosteal cells from a donor, from which the MSCs are subsequently isolated.

Umbilical cord blood (UCB) is a known alternative source of hematopoietic progenitor stem cells. Conventional techniques for the collection of UCB are based on the use of a needle or cannula, which is used with the aid of gravity to drain cord blood from (i.e., exsanguinate) the placenta (see also Anderson, U.S. Pat. No. 5,372,581 and Hessel et al, U.S. Pat. No. 5,415,665). The needle or cannula is usually placed in the umbilical vein and the placenta is gently massaged to aid in draining cord blood from the placenta.

The cells so obtained can either be used directly or preserved. For example, stem cells from cord blood are routinely cryopreserved for use in hematopoietic reconstitution, a widely used therapeutic procedure used in bone marrow and other related transplantations (see e.g., Boyse et al., U.S. Pat. No. 5,004,681 and Boyse et al., U.S. Pat. No. 5,192,553).

Erices et al., in Br. J. Haematology 109: 235-42, 2000, describe a pluripotent cell derived from human cord blood. Naughton et al. (U.S. Pat. No. 5,962,325) describe fetal pluripotent cells, including fibroblast-like cells and chondrocyte-progenitors, obtained from umbilical cord or placenta tissue or umbilical cord blood. The fetal stromal cells so obtained can be used to prepare a “generic” stromal or cartilaginous tissue. Naughton et al. also disclose that a “specific” stromal tissue may be prepared by inoculating a three-dimensional matrix with fibroblasts derived from a particular individual who is later to receive the cells and/or tissues grown in culture in accordance with the disclosed methods.

Methods are known for the clonogenic expansion and selection of pluripotent cells derived from cord blood. Kraus et al. (U.S. Pat. No. 5,674,750) describe a system for growing relatively undifferentiated cells on the surface of beads that bear a surface antigen recognized by the pluripotent cell. Kraus et al. (U.S. Pat. Nos. 5,925,567 and 6,338,942) provide additional methods for selecting for predetermined target cell populations of pluripotent cells. In one example, a starting sample of cells from cord blood or peripheral blood are introduced into a growth medium, causing cells of the target cell population to divide, followed by contacting the cells in the growth medium with a selection element with affinity for a predetermined population of cells to select cells of the predetermined target population from other cells in the growth medium.

Methods exist for the isolation, preservation, propagation, differentiation, and selection of pluripotent cells derived from umbilical cord blood or placental blood; these cells can be used in a variety of therapeutic methods for the treatment of disease.

### SUMMARY OF THE INVENTION

In a first aspect, the invention features the use of pluripotent cells, such as those progenitor cells isolated from UCB described by Erices et al., in Br. J. Haematology 109: 235-42, 2000, to treat a vascular, a muscle, a hepatic, a pancreatic, or a neural disease that includes the step of administering to a patient a pluripotent cell derived from human umbilical cord blood, placental blood, and/or a blood sample from a newborn, or administering to the patient a progeny cell of the pluripotent cell, wherein the pluripotent cell expresses SH2, SH3, SH4, CD13, CD29, CD49e, CD54, and CD90 antigen markers; does not express CD14, CD31, CD34, CD45, CD49d, and CD106 antigen markers; and is capable of differentiating into mesenchymal pluripotent cells, hematopoietic pluripotent cells, neural pluripotent cells, or endothelial pluripotent cells. In one embodiment, the method includes organ regeneration. In another embodiment, the method includes the in vitro growth of blood vessels, which can then be used, for example, for the replacement of damaged blood vessels in the patient.

In another embodiment, the method further includes inducing a progeny of the pluripotent cell to express an endothelial cell marker, preferably expressing a marker recognized by the P1H12 monoclonal antibody; a liver cell marker; a pancreatic cell marker; a cardiac or smooth muscle cell marker; or a nerve cell marker before administration of the progeny cell to the patient. In another embodiment, the pluripotent cells, or their progeny, are induced to differentiate into a cell type that can be used for wound or vessel repair or to regenerate wounded or damaged tissue.

In another aspect, the invention features a method of identifying an agent that is capable of inducing differentiation of an isolated pluripotent cell. The method involves contacting the pluripotent cell, which is characterized by the expression of SH2, SH3, SH4, CD13, CD29, CD49e, CD54, and CD90 antigens and the absence of expression of CD14, CD34, CD45, CD49d, and CD106 antigens, with a test agent, followed by the detection of a change in marker expression of the contacted pluripotent cell, relative to a pluripotent cell that is not contacted with the test agent, wherein a change in marker expression indicates that the test agent induces differentiation of the pluripotent cell. The method further comprises determining whether the test agent promotes the differentiation of the pluripotent cell into an endothelial cell marker, a liver cell marker, a pancreatic cell marker, a cardiac or smooth muscle cell marker, or a nerve cell marker, by detecting the presence of one or more markers specific to the desired cell type.

In another aspect, the invention features a method for producing a population of cells characterized by the expression of SH2, SH3, SH4, CD13, CD29, CD49e, CD54, and CD90 antigen markers, and the absence of expression of CD14, CD34, CD45, CD49d, and CD106 antigen markers that includes the steps of (a) providing pluripotent cells derived from umbilical cord blood and capable of differentiating into mesenchymal pluripotent cells, hematopoietic pluripotent cells, neural pluripotent cells, or endothelial pluripotent cells; (b) culturing the pluripotent cells of step (a) in a medium containing dexamethasone for a time sufficient to expand the population of pluripotent cells; and (c) isolating the pluripotent cells from the culture, wherein greater than 20% of said isolated pluripotent cells are positive for SH2, SH3, SH4, CD13, CD29, CD49e, CD54, and CD90 markers, and negative for CD14, CD34, CD45, CD49d, and CD106 markers.

In another aspect, the invention features a composition comprising pluripotent cells that are positive for SH2, SH3, SH4, CD13, CD29, CD49e, CD54, and CD90 markers and negative for CD14, CD34, CD45, CD49d, and CD106 markers, and a pharmaceutically acceptable carrier.

In another aspect, the invention features a pluripotent progeny cell obtained from the in vitro or ex vivo transformation of a pluripotent cell positive for SH2, SH3, SH4, CD13, CD29, CD49e, CD54, and CD90 markers and negative for CD14, CD34, CD45, CD49d, and CD106 markers. In an embodiment of this aspect, the transformed progeny cell can be part of a composition that also includes a pharmaceutically acceptable carrier. In all aspects of the invention, the pharmaceutically acceptable carrier can be saline, a gel, a hydrogel, a sponge, or a matrix.

In another aspect, the invention features a method of gene therapy that includes administering to a patient a transformed progeny cell derived from pluripotent cells obtained from UCB that are positive for SH2, SH3, SH4, CD13, CD29, CD49e, CD54, and CD90 markers and negative for CD14, CD34, CD45, CD49d, and CD106 markers, in which the progeny cell expresses a gene of interest (e.g., a therapeutic protein, such as a growth factor or matrix molecule).

In another aspect, the invention features a method for providing a patient with a therapeutic protein that includes administering to the patient a transformed progeny cell derived from pluripotent cells obtained from UCB that are positive for SH2, SH3, SH4, CD13, CD29, CD49e, CD54, and CD90 markers and negative for CD14, CD34, CD45, CD49d, and CD106 markers, in which the progeny cells have been transformed with a DNA molecule encoding the therapeutic protein, such that the progeny cells express a therapeutically effective amount of the therapeutic protein in the patient.

By a “neural cell” is meant a neuron (e.g., a sensory neuron, a motor neuron, or an interneuron) or a support cell of the central or peripheral nervous system. Examples of neurons include pyramidal cells, Betz cells, stellate cells, horizontal cells, granule cells, Purkinje cells, spinal motor neurons, and ganglion cells. Examples of support cells include glial cells, oligodendroglial cells, astrocytes, satellite cells, microglial cells, and Schwann cells.

By a “muscle cell” is meant a skeletal, smooth, or cardiac cell.

By a “vascular cell” is meant an endothelial cell. Endothelial cells line the blood and lymph vessels and are present in and play a key role in the development of organs, such as the brain, heart, liver, pancreas, lungs, spleen, stomach, intestines, and kidneys.

By “umbilical cord blood cells”, “cord blood cells”, or “placental blood cells” we mean the blood that remains in the umbilical cord and placenta following birth. Like bone marrow, cord blood has been found to be a rich source of cord cells.

By “stem cell” or “pluripotent cell,” which can be used interchangeably, is meant a cell having the ability to give rise to two or more cell types of an organism.

A molecule is a “marker” of a desired cell type if it is found on a sufficiently high percentage of cells of the desired cell type, and found on a sufficiently low percentage of cells of an undesired cell type, such that one can achieve a desired level of purification of the desired cell type from a population of cells comprising both desired and undesired cell types by selecting for cells in the population of cells that have the marker. A marker can be displayed on, for example, 30%, 35%, 40%, 45%, 50%, 55%, 60%, 65%, 70%, 75%, 80%, 85%, 90%, 95%, 99% or more of the desired cell type, and can be displayed on fewer than 50%, 45%, 40%, 35%, 30%, 25%, 20%, 15%, 10%, 5%, 1% or fewer of an undesired cell type.

A desired cell type is negative for a cell surface-expressed marker or lacks expression of the marker if fewer than 50 marker molecules per cell are present on the cell surface of the desired cell type. Techniques for detecting cell surface-expressed marker molecules are well known in the art and include, e.g., flow cytometry. One skilled in the art can also use enzymatic amplification staining techniques in conjunction with flow cytometry to distinguish between cells expressing a low number of a marker molecule and cells that do not express the marker molecule (see, e.g., Kaplan, Front. Biosci. 7:c33-c43, 2002; Kaplan et al., Amer. J. Clin. Pathol. 116:429-436, 2001; and Zola et al., J. Immunol Methods 135:247-255, 1990).

By “neural disease” is meant a disease or disorder that affects or involves the central or peripheral nervous system. Examples of neural diseases include multi-infarct dementia (MID), vascular dementia, cerebrovascular injury, Alzheimer's disease (AD), neurofibromatosis, Huntington's disease, amyotrophic lateral sclerosis, multiple sclerosis, stroke, Parkinson's disease (PD), pathologies of the developing nervous system, pathologies of the aging nervous system, and trauma, e.g., head trauma. Other examples of neural diseases are those that affect tissues of the eye, e.g., the optic stalk, retinal layer, and lens of the eye, and the inner ear. In certain embodiments, the patient may have suffered a neurodegenerative disease, a traumatic injury, a neurotoxic injury, ischemia, a developmental disorder, a disorder affecting vision, an injury or disease of the spinal cord, or a demyelinating disease.

By “muscle disease” is meant a disease or disorder that affects or involves the musculature, e.g., cardiac, smooth, or skeletal muscles. Examples of muscle diseases include neuromuscular disease, e.g., muscular dystrophy (e.g., Duchenne muscular dystrophy (DMD), Becker muscular dystrophy (BMD), Limb-girdle muscular dystrophy, and congenital muscular dystrophy), congenital myopathy, and myasthenia gravis, cardiomyopathy, e.g., heart disease, aortic aneurysm (Marfan's disease), cardiac ischemia, congestive heart failure, heart valve disease, and arrhythmia, and metabolic muscle diseases.

By “vascular disease” is meant a disease or disorder that affects or involves the vasculature. Examples of vascular disease include peripheral vascular disease, peripheral arterial disease, venous disease (e.g., deep vein thrombosis), ischemia, cardiovascular disease, tissue organ engraftment rejection, or sequelae of ischemic reperfusion injury. In still another embodiment, the peripheral vascular disease is atherosclerosis, thromboembolic disease, or Buerger's disease (thromboangiitis obliterans). In a further embodiment, the cardiovascular disease is myocardial infarction, heart disease, or coronary artery disease.

### DETAILED DESCRIPTION

The pluripotent cells used in the methods and in the compositions of the invention can be from a spectrum of sources including, in order of preference: autologous, allogeneic, or xenogeneic sources. The pluripotent cells of the invention can be isolated and purified by several methods, including the steps of density gradient isolation and culture of adherent cells as described in Example 1. After a confluent cell layer has been established, the isolation process to derive cells of this invention is routinely controlled by morphology (fibroblastoid morphology) and phenotypical analyses using antibodies directed against SH2 (positive), SH3 (positive), SH4 (positive), CD13 (positive), CD29 (positive), CD49e (positive), CD54 (positive), CD90 (positive), CD14 (negative), CD31 (negative), CD34 (negative), CD45 (negative), CD49d (negative), and CD106 (negative) markers (see Example 2).

The methods of the invention use a pluripotent cell that reacts negatively with markers specific for the hematopoietic lineage, such as CD45, and hence, is distinct from hematopoietic stem cells which can also be isolated from placental cord blood. CD14 is another surface antigen that cannot be detected on the pluripotent cells used in the methods of the invention. Typically, the pluripotent cells useful for the practice of the invention exhibit fibroblastoid cell shape and proliferate in an adherent manner.

The pluripotent cell used in the methods of the invention can be present in a plurality or mixtures representing precursors of other stem cells, e.g., of the haematopoietic lineage preferably expressing AC133 and CD34, mesenchymal stem cells, neuronal stem cells, endothelial stem cells, or combinations thereof. Preferably, the other stem cells of the mixture are progeny of cells that express SH2, SH3, SH4, CD13, CD29, CD49e, CD54, and CD90 antigen markers, but do not express CD14, CD31, CD34, CD45, CD49d, and CD106 antigen markers.

### Organ/Tissue Regeneration

The pluripotent cells of the invention or their progeny can be used in a variety of applications. These include, but are not limited to, transplantation or implantation of the cells either in unattached form or as attached, for example, to a three-dimensional framework, as described herein. Typically, 10² to 10⁹ cells are transplanted in a single procedure, with additional transplants performed as required. The tissue produced according to the methods of the invention can be used to repair or replace damaged or destroyed tissue, to augment existing tissue, to introduce new or altered tissue, to modify artificial prostheses, or to join biological tissues or structures.

If the pluripotent cells are derived from a heterologous source relative to the recipient subject, concomitant immunosuppression therapy can be administered, e.g., administration of the immunosuppressive agent cyclosporine or FK506. However, due to the immature state of pluripotent cells derived from UCB, such immunosuppressive therapy may not be required. Accordingly, in one example, pluripotent mesenchymal cells derived from UCB can be administered to a recipient in the absence of immunomodulatory (e.g., immunsuppressive) therapy.

In addition, injection of extracellular matrix prepared from new tissue produced by pluripotent cells derived from UCB, or their progeny, can be administered to a subject or may be used to further culture cells. Such cells, tissues, and extracellular matrix may serve to repair, replace or augment endothelial tissue that has been damaged due to disease or trauma, or that failed to develop normally, or for cosmetic purposes.

A formulation of pluripotent mesenchymal cells derived from UCB or their progeny can be injected or administered directly to the site where the production of new tissue is desired. For example, and not by way of limitation, the pluripotent cells may be suspended in a hydrogel solution for injection. Alternatively, the hydrogel solution containing the cells may be allowed to harden, for instance in a mold (e.g., a vascular or tubular tissue construct), to form a matrix having cells dispersed therein prior to implantation. Once the matrix has hardened, the cell formations may be cultured so that the cells are mitotically expanded prior to implantation. A hydrogel is an organic polymer (natural or synthetic) which is cross-linked via covalent, ionic, or hydrogen bonds to create a three-dimensional open-lattice structure, which entraps water molecules to form a gel. Examples of materials which can be used to form a hydrogel include polysaccharides such as alginate and salts thereof, polyphosphazines, and polyacrylates, which are cross-linked ionically, or block polymers such as PLURONICS™ or TETRONICS™ (BASF Corp., Mount Olive, N.Y.), polyethylene oxide-polypropylene glycol block copolymers which are cross-linked by temperature or pH. Methods of synthesis of the hydrogel materials, as well as methods for preparing such hydrogels, are known in the art.

Such cell formulations may further comprise one or more other components, including selected extracellular matrix components, such as one or more types of collagen known in the art, and/or growth factors and drugs. Growth factors which may be usefully incorporated into the cell formulation include one or more tissue growth factors known in the art or to be identified in the future, such as but not limited to any member of the TGF-β family, IGF-I and -II, growth hormone, BMPs such as BMP-13, and the like. Alternatively, pluripotent mesenchymal cells derived from UCB may be genetically engineered to express and produce growth factors such as BMP-13 or TGF-β. Details on genetic engineering of the cells of the invention are provided herein. Drugs that may be usefully incorporated into the cell formulation include, for example, anti-inflammatory compounds, as well as local anesthetics. Other components that may also be included in the formulation include, for example, buffers to provide appropriate pH and isotonicity, lubricants, viscous materials to retain the cells at or near the site of administration, (e.g., alginates, agars, and plant gums) and other cell types that may produce a desired effect at the site of administration (e.g., enhancement or modification of the formation of tissue or its physicochemical characteristics, support for the viability of the cells, or inhibition of inflammation or rejection).

Pluripotent mesenchymal cells derived from UCB can be administered directly and induced to differentiate by contact with tissue in vivo or induced to differentiate into a desired cell type, e.g., mesenchymal cells, hematopoietic cells, neural cells, or endothelial cells, etc., using in vitro or ex vivo methods before their administration. Such predisposition of progeny of pluripotent mesenchymal cells derived from UCB has the potential to shorten the time required for complete differentiation once the cells have been administered to the patient. Techniques for the differentiation of pluripotent cells into cells of a particular phenotype are known in the art, such as those described in U.S. Pat. Nos. 5,486,359; 5,591,625; 5,736,396; 5,811,094; 5,827,740; 5,837,539; 5,908,782; 5,908,784; 5,942,225; 5,965,436; 6,010,696; 6,022,540; 6,087,113; 5,858,390; 5,804,446; 5,846,796; 5,654,186; 6,054,121; 5,827,735; and 5,906,934, which describe the transformation of pluripotent cells. For example, Rodgers et al. (U.S. Pat. No. 6,335,195), describes methods for the ex vivo culturing of hematopoietic and mesenchymal pluripotent cells and the induction of lineage-specific cell proliferation and differentiation by growth in the presence of angiotensinogen, angiotensin I (AI), AI analogues, AI fragments and analogues thereof, angiotensin II (AII), AII analogues, AII fragments or analogues thereof, or AII AT₂-type 2 receptor agonists, either alone or in combination with other growth factors and cytokines. In an embodiment, the pluripotent cells of the invention can be induced in vitro to differentiate into pancreatic cells, and in particular pancreatic islet cells, by using, e.g., techniques known in the art (see, e.g., Yang et al., Proc. Nat. Acad. Sci. USA 99: 8078-83, 2002; Zulewski et al., Diabetes 50: 521-33, 2001; and Bonner-Weir et al., Proc. Nat. Acad. Sci. USA 97: 7999-8004, 2001). Art-known techniques can also be used to induce the pluripotent cells of the invention to differentiate in vitro into hepatic cells (see, e.g., Lee et al., Hepatology 40: 1275-1284, 2004), neuronal cells (see, e.g., Thondreau et al., Differentiation 319-322-326, 2004), or endothelial cells (see, e.g., Kassem et al., Basic Clin. Pharmacol. & Toxicol. 95:209-214, 2004; and Pittenger and Martin, Circ. Res. 95:9-20, 2004). Optionally, a differentiating agent may be co-administered or subsequently administered to the subject to promote stem cell differentiation in vivo.

Pluripotent mesenchymal cells derived from UCB or their progeny can be used to produce new tissue in vitro, which can then be implanted, transplanted, or otherwise inserted into a site requiring tissue repair, replacement, or augmentation in a subject. Pluripotent mesenchymal cells derived from UCB or their progeny may be inoculated or “seeded” onto a three-dimensional framework or scaffold, and proliferated or grown in vitro to form a living endothelial tissue which can be implanted in vivo. The three-dimensional framework may be of any material and/or shape that allows cells to attach to it (or can be modified to allow cells to attach to it) and allows cells to grow in more than one layer. A number of different materials may be used to form the matrix, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), nitrocellulose, cotton, polyglycolic acid (PGA), collagen (in the form of sponges, braids, or woven threads, and the like), cat gut sutures, cellulose, gelatin, or other naturally occurring biodegradable materials or synthetic materials, including, for example, a variety of polyhydroxyalkanoates. Any of these materials may be woven into a mesh, for example, to form the three-dimensional framework or scaffold. The pores or spaces in the matrix can be adjusted by one of skill in the art to allow or prevent migration of cells into or through the matrix material. In one example, Naughton et al. (U.S. Pat. No. 6,022,743), describe a tissue culture system in which stem cells or progenitor cells (e.g., stromal cells such as those derived from umbilical cord cells, placental cells, mesenchymal stem cells or fetal cells) are propagated on three-dimensional supports.

The three-dimensional framework, matrix, hydrogel, and the like, can be molded into a form suitable for the tissue to be replaced or repaired. For example, where a vascular graft is desired, the three-dimensional framework can be molded in the shape of a tubular structure and seeded with endothelial stem cells of the invention alone or in combination with stromal cells (e.g., fibroblasts) and cultured accordingly. In addition to pluripotent cells derived from UCB, or their progeny, other cells may be added to the three-dimensional framework so as to improve the growth of, or alter, one or more characteristics of the new tissue formed thereon. Such cells may include, but are not limited to, fibroblasts, pericytes, macrophages, monocytes, plasma cells, mast cells, and adipocytes, among others.

Alternatively, the cells can be encapsulated in a device or microcapsule, which permits exchange of fluids but prevents cell/cell contact. Transplantation of microencapsulated cells is known in the art, e.g., Balladur et al., Surgery 117: 189-94, 1995; and Dixit et al., Cell Transplantation 1: 275-79, 1992. In one example, the cells may be contained in a device which is viably maintained outside the body as an extracorporeal device. Preferably, the device is connected to the blood circulation system such that the pluripotent cells can be functionally maintained outside of the body and serve to assist organ failure conditions. In another example, the encapsulated cells may be placed within a specific body compartment such that they remain functional for extended periods of time in the absence or presence of immunosuppressive or immuno-modulatory drugs.

In yet another example, pluripotent mesenchymal cells derived from UCB or their progeny can be used in conjunction with a three-dimensional culture system in a “bioreactor” to produce tissue constructs which possess critical biochemical, physical and structural properties of native human tissue by culturing the cells and resulting tissue under environmental conditions which are typically experienced by the native tissue. Thus, the three-dimensional culture system may be maintained under intermittent and periodic pressurization and the cells of the invention provided with an adequate supply of nutrients by convection. Maintaining an adequate supply of nutrients to the cells of the invention throughout a replacement endothelial tissue construct of approximately 2-5 mm thickness is important as the apparent density of the construct increases. Pressure facilitates flow of fluid through the three-dimensional endothelial construct, thereby improving the supply of nutrients and removal of waste from cells embedded in the construct. The bioreactor may include a number of designs. Typically the culture conditions will include placing a physiological stress on the construct containing cells similar to what will be encountered in vivo. For example, the vascular construct may be cultured under conditions that simulate the pressures and shear forces of blood vessels (see, for example, U.S. Pat. No. 6,121,042, which is hereby incorporated by reference herein).

The methods of the invention may be used to treat subjects requiring the repair or replacement of endothelial tissue resulting from disease or trauma, or to provide a cosmetic function, such as to augment facial or other features of the body. Treatment may entail the in vivo use of pluripotent mesenchymal cells derived from UCB or their progeny to produce new endothelial tissue, or the use of the endothelial tissue produced in vitro or ex vivo, according to any method presently known in the art or to be developed in the future. For example, pluripotent cells derived from UCB, or tissue derived from the isolated pluripotent cells, may be implanted, injected, or otherwise administered directly to the site of tissue damage so that they will produce new endothelial tissue in vivo.

In another example, the methods of the invention would include the replacement of a heart valve prepared with pluripotent mesenchymal cells derived from UCB or their progeny and vascular tissue or graft. In another example, pluripotent mesenchymal cells derived from UCB or their progeny are administered in combination with angiogenic factors to induce or promote new capillary or vessel formation in a subject. By “angiogenic factor” is meant a growth factor, protein or agent that promotes or induces angiogenesis in a subject. The cells of the invention can be administered prior to, concurrently with, or following injection of the angiogenic factor. In addition, pluripotent mesenchymal cells derived from UCB may be administered immediately adjacent to, at the same site, or remotely from the site of administration of the angiogenic factor.

As cardiac muscle does not normally have reparative potential, pluripotent mesenchymal cells derived from UCB or their progeny can be used to regenerate or repair striated cardiac muscle that has been damaged through disease or degeneration. In such a therapy, the pluripotent cells differentiate into cardiac muscle cells and integrate with the healthy tissue of the recipient to replace the function of the dead or damaged cells, thereby regenerating the cardiac muscle as a whole. The pluripotent cells are used, for example, in cardiac muscle regeneration for a number of principal indications: (i) ischemic heart implantations, (ii) therapy for congestive heart failure patients, (iii) prevention of further disease in patients undergoing coronary artery bypass graft, (iv) conductive tissue regeneration, (v) vessel smooth muscle regeneration, and (vi) valve regeneration.

Pluripotent cell therapy for heart-related disease is based, for example, on the following sequence: harvesting of pluripotent cells derived from UCB, isolation/expansion of the pluripotent cells, implantation into the damaged heart (with or without a stabilizing matrix and biochemical manipulation), and in situ formation of myocardium. This approach is different from traditional tissue engineering, in which the tissues are grown ex vivo and implanted in their final differentiated form. Biological, bioelectrical and/or biomechanical triggers from the host environment may be sufficient, or under certain circumstances, may be augmented as part of the therapeutic regimen to establish a fully integrated and functional tissue.

Pluripotent mesenchymal cells derived from UCB or their progeny can be useful in the treatment of pancreatic or hepatic diseases or disorders. For example, pluripotent mesenchymal cells derived from UCB may be implanted, injected, or otherwise administered directly to the site of damage so that they will produce new pancreatic or hepatic tissue in vivo. Methods of treatment include identifying a patient having a extraintestinal gastrointestinal or a hepaticopancreatic disorder and administering to the patient a therapeutically effective amount of a composition that includes pluripotent mesenchymal cells derived from UCB or their progeny to treat the disorder. An “extraintestinal gastrointestinal” disorder is a disorder of the gastrointestinal tract that is primarily localized in an area other than the interior of the intestine. Non-limiting examples of extraintestinal gastrointestinal disorders include hepaticopancreatic disorders, duodenum disorders, bile duct disorders, appendix disorders, spleen disorders, and stomach disorders. “Hepaticopancreatic” disorders are disorders of the pancreas and liver. Non-limiting examples of hepaticopancreatic disorders include diabetes, pancreatitis, hepatic cirrhosis, hepatitis, cancer and pancreatico-biliary disease. A “disorder” of a particular organ or structure includes situations where the organ or structure is entirely absent. For example, for the purposes of this invention, a person who lacks a pancreas has a pancreas disorder. Methods of implanting exogenous tissue are well known (see, e.g., J. Shapiro et. al., New Engl J. Med. 343: 230-238, 2000, for the transplantation of pancreatic islets).

Pluripotent mesenchymal cells derived from UCB or their progeny can be useful in the treatment of neural diseases. In one example, the pluripotent cells are administered to a patient to affect neurogenesis or gliogenesis in the central nervous system, such as the brain. Such treatment may be aimed at patients with Parkinson's disease, Alzheimer's disease, or who have suffered a stroke or trauma. In the case of glial cells, the therapy may be intended for treating multiple sclerosis and other glia related conditions. Other examples of tissues that could be generated are the optic stalk, retinal layer, and lens of the eye, and the inner ear. In certain methods, the patient may have suffered a neurodegenerative disease, a traumatic injury, a neurotoxic injury, ischemia, a developmental disorder, a disorder affecting vision, an injury or disease of the spinal cord, or a demyelinating disease. These patients having a neural disease or disorder that may be associated with impaired function can be administered a pharmaceutically effective amount of pluripotent cells that produce neurons, or other cell type depending on the neural disease or disorder to be treated.

### In Vitro/Ex Vivo Use of UCB-Derived Pluripotent Mesenchymal Cells

Pluripotent mesenchymal cells derived from UCB or their progeny can be used in vitro to screen for the efficacy and/or cytotoxicity of compounds, allergens, growth/regulatory factors, pharmaceutical compounds, and the like on endothelial stem cells, to elucidate the mechanism of certain diseases by determining changes in the biological activity of the pluripotent cells (e.g., proliferative capacity, adhesion), to study the mechanism by which drugs and/or growth factors operate to modulate endothelial stem cell biological activity, to diagnose and monitor cancer in a patient, for gene therapy, gene delivery or protein delivery, and to produce biologically active products.

Pluripotent cells derived from UCB, or progeny thereof may be used in vitro to screen a wide variety of agents for effectiveness and cytotoxicity of pharmaceutical agents, growth/regulatory factors, anti-inflammatory agents, and the like. To this end, the pluripotent cells can be maintained in vitro and exposed to the agent to be tested. The activity of a cytotoxic agent can be measured by its ability to damage or kill the pluripotent cells or their progeny in culture. This can be assessed readily by utilizing a cell viability assay, such as a staining technique (e.g., trypan blue staining). The effect of growth/regulatory factors can be assessed by analyzing the number of living cells in vitro, e.g., by total cell counts, and differential cell counts. This can be accomplished using standard cytological and/or histological techniques, including the use of immunocytochemical techniques employing antibodies that define type-specific cellular antigens. The effect of various drugs on UCB-derived pluripotent cells can be assessed either in a suspension culture or in a three-dimensional system.

Pluripotent mesenchymal cells derived from UCB can also be used in the isolation and evaluation of factors associated with the differentiation and maturation of mesenchymal cells, hematopoietic cells, neural cells, or endothelial cells. Thus, the pluripotent cells of the invention may be used in assays to evaluate fluids, such as media, e.g., conditioned media, for the presence of a factor capable of promoting cell growth, e.g., the growth of mesenchymal cells, hematopoietic cells, neural cells, or endothelial cells, and the like. The pluripotent cells of the invention can also be used to identify factors capable of promoting the differentiation and/or maturation of a cell type, e.g., mesenchymal cells, hematopoietic cells, neural cells, or endothelial cells, to a particular lineage. Various systems are applicable and can be designed to induce differentiation of the stem cells based upon various physiological stresses. For example, a bioreactor system can be employed with the cells of the present invention, e.g., a bioreactor that simulates vascular tissue.

### Gene Therapy

Genetically altered pluripotent cells are useful to produce both non-therapeutic and therapeutic recombinant proteins in vivo and in vitro. Pluripotent mesenchymal cells derived from UCB can be isolated from a donor (non-human or human) as described in Example 1, transfected or transformed with a recombinant polynucleotide in vitro or ex vivo, and transplanted into the recipient or cultured in vitro. The genetically altered pluripotent cells or progeny can then produce the desired recombinant protein in vivo or in vitro. The produced protein or molecule may have direct or indirect therapeutic usefulness, or it may have usefulness as a diagnostic protein or molecule.

Therapeutic uses of pluripotent mesenchymal cells derived from UCB that have been genetically transformed include transplanting the pluripotent cells, pluripotent cell populations, or progeny thereof into individuals to treat a variety of pathological states including diseases and disorders resulting from myocardial damage, circulatory or vascular disorders or diseases, neural diseases or disorders, hepatic diseases or disorders, or pancreatic diseases or disorders, as well as tissue regeneration and repair. By the same techniques described above, the genetically altered pluripotent cells or pluripotent cell populations used in the methods of the invention can be administered to a subject in need of such cells or in need of the protein or molecule encoded or produced by the genetically altered cell.

For example, genes that express products capable of preventing or ameliorating symptoms of various types of diseases or disorders (e.g., vascular diseases or disorders) or that prevent or promote inflammatory disorders can be incorporated into pluripotent cells derived from UCB. In one example, these pluripotent cells are genetically engineered to express an anti-inflammatory gene product that would serve to reduce the risk of failure of implantation or further degenerative change in tissue due to inflammatory reaction. The expression of one or more anti-inflammatory gene products include, for example, peptides or polypeptides corresponding to the idiotype of antibodies that neutralize granulocyte-macrophage colony stimulating factor (GM-CSF), TNF-α, IL-1, IL-2, or other inflammatory cytokines. IL-1 has been shown to decrease the synthesis of proteoglycans and collagens type II, IX, and XI (Tyler et al., Biochem. J. 227: 69-878, 1985; Tyler et al., Coll. Relat. Res. 82: 393-405, 1988; Goldring et al., J. Clin. Invest. 82: 2026-2037, 1988; and Lefebvre et al., Biophys. Acta. 1052: 366-72, 1990). TNF-α also inhibits synthesis of proteoglycans and type II collagen, although it is much less potent than IL-1 (Yaron et al., Arthritis Rheum. 32: 173-80, 1989; Ikebe et al., J. Immunol. 140: 827-31, 1988; and Saklatvala Nature 322: 547-49, 1986). Also, for example, pluripotent mesenchymal cells derived from UCB may be engineered to express the gene encoding the human complement regulatory protein that prevents rejection of a graft by the host. See, for example, McCurry et al., Nature Medicine 1: 423-27, 1995. In another example, pluripotent mesenchymal cells derived from UCB can be engineered to include a gene or polynucleotides sequence that expresses or causes to be expressed an angiogenic factor.

Alternatively, pluripotent mesenchymal cells derived from UCB may be genetically engineered to express and produce growth factors such as VEGF, FGF, EGF, IGF, as well as therapeutic agents such as TWEAK, TWEAKR, TNFR, other anti-inflammatory agents, or angiogenic agents. For example, the gene or coding sequence for such growth factors or therapeutic agents would be placed in operative association with a regulated promoter so that production of the growth factor or agent in culture can be controlled.

In another example, pluripotent mesenchymal cells derived from UCB are genetically modified or engineered to contain genes which express proteins of importance for the differentiation and/or maintenance of striated cardiac muscle cells. Examples include growth factors (TGF-β, IGF-1, FGF), myogenic factors (myoD, myogenin, Myf5, MRF), transcription factors (GATA-4), cytokines (cardiotrophin-1), members of the neuregulin family (neuregulin 1, 2 and 3) and homeobox genes (Csx, tinman, NKx family).

Alternatively, the transformed pluripotent cells may be genetically engineered to “knock out” expression of native gene products that promote inflammation, e.g., GM-CSF, TNF, IL-1, IL-2, or “knock out” expression of MHC in order to lower the risk of rejection. In addition, the cells may be genetically engineered for use in gene therapy to adjust the level of gene activity in a subject to assist or improve the results of a transplantation.

Genetically engineered pluripotent cells may also be screened to select those cell lines that bring about the amelioration of symptoms of rheumatoid disease or inflammatory reactions in vivo, and/or escape immunological surveillance and rejection.

Conventional recombinant DNA techniques are used to introduce the desired polynucleotide into the pluripotent cells or their progeny. For example, physical methods for the introduction of polynucleotides into cells include microinjection and electroporation. Chemical methods such as coprecipitation with calcium phosphate and incorporation of polynucleotides into liposomes are also standard methods of introducing polynucleotides into mammalian cells. For example, DNA or RNA can be introduced using standard vectors, such as those derived from murine and avian retroviruses (see, e.g., Gluzman et al, Viral Vectors, 1988, Cold Spring Harbor Laboratory, Cold Spring Harbor, N.Y.). Standard recombinant molecular biology methods are well known in the art (see, e.g., Ausubel et al., Current Protocols in Molecular Biology, 1989, John Wiley & Sons, New York), and viral vectors for gene therapy have been developed and successfully used clinically (Rosenberg et al., N. Engl. J. Med., 323: 370, 1990). Other methods, such as naked polynucleotide uptake from a matrix coated with DNA are also encompassed by the invention (see, for example, U.S. Pat. No. 5,962,427, which is incorporated herein by reference).

Pluripotent mesenchymal cells derived from UCB that have been genetically modified can be cultured in vitro to produce biological products in high yield. For example, such cells, which either naturally produce a particular biological product of interest (e.g., a growth factor, regulatory factor, or peptide hormone, and the like), or have been genetically engineered to produce a biological product, could be clonally expanded. If the cells secrete the biological product into the nutrient medium, the product can be readily isolated from the spent or conditioned medium using standard separation techniques, e.g., such as differential protein precipitation, ion-exchange chromatography, gel filtration chromatography, electrophoresis, and HPLC, to name but a few. Alternatively, a biological product of interest may remain within the cell and, thus, its collection may require lysis of the cells. The biological product may then be purified using any one or more of the above-listed techniques.

### Administration of UCB-derived Pluripotent Mesenchymal Cells by Systemic Infusion

Pluripotent cells of the invention are prepared and isolated as described above. The pluripotent cells, or expanded sub-populations of these cells, can be administered to a patient in need using one or more methods known in the art. For example, the pluripotent cells can be administered by infusion into the patient by, e.g., intracoronary infusion, retrograde venous infusion (see, e.g., Perin and Silva, Curr. Opin. Hematol. 11:399-403, 2004), intraventricular infusion, intracerebroventricular infusion, cerebrospinal infusion, and intracranial infusion. The administration of cells by infusion may need to be repeated one or more times during treatment. If multiple infusions of cells are performed, the infusions can be administered over time, e.g., one on day one, a second on day five, and a third on day ten. After the initial ten-day period, there can be a period of time without cell administration, e.g., two weeks to 6 months, after which time the ten-day administration protocol can be repeated.

### Administration of UCB-Derived Pluripotent Mesenchymal Cells by Direct Injection

Another possible administration route for the pluripotent cells of the invention, or expanded sub-populations of these cells, is via direct surgical injection (e.g., intramyocardial or transendocardial injection, intracranial, intracerebral, or intracisternal injection, intramuscular injection, intrahepatic injection, and intrapancreatic injection) into the tissue or region of the body to be treated (e.g., the brain, muscle, heart, liver, pancreas, and vasculature). This method of administration may also require multiple injections with treatment interruption intervals lasting from 2 week to 6 months, or as otherwise determined by the attending physician.

Administration of UCB-derived Pluripotent Mesenchymal Cells by Implantation

The UCB-derived pluripotent mesenchymal cells can also be administered by implantation into a patient at the site of disease or injury or at a site that will facilitate treatment of the disease or injury.

The invention is further described in the following non-limiting examples.

### Example 1

### Collection and Isolation of Pluripotent Cells Derived from Umbilical Cord Blood (UCB)

Collection of cord blood is performed with the informed consent of the mother. After delivery of a baby with the placenta still in utero, the umbilical cord is doubly clamped and transsected 7-10 cm away from the umbilicus. The blood is allowed to drain from the severed end of the cord into bottles containing 10 mL of M-199 culture medium with 250 U/mL of preservative-free heparin. In all cases, blood samples are processed within 24 hours after harvest. From each blood harvest, aliquots are set apart for routine haematological analysis (Cell-Dyn 3500 System, Abbott) and for immunophenotyping of haematopoietic progenitors.

Cord blood cells are separated into a low-density fraction (Hystopaque-1077; Sigma, St. Louis, USA) and mononuclear cells are washed, suspended in culture medium ([alpha]-MEM, USA) and seeded (T-25 flasks and 35 mm dishes) at a concentration of 1×10⁶ cells/cm². Cultures are maintained at 37° C. in a humidified atmosphere containing 5% CO₂, with a change of culture medium every 7 days. Cells in the developing adherent layer are used for the examples below. An example of the generation of adherent stem cells can be found in Beerheide et al., Biochem. Biophys. Res Comm. 294: 1052-63, 2002.

### Example 2

Immunophenotping of Cells by Cytofluorometry

To detect surface antigens, aliquots of fresh UCB cells, or cultured adherent cells that have been detached with 0.25% EDTA, are washed with phosphate-buffered saline (PBS) containing 2% FBS. To detect intracellular antigens, cultured adherent cells are detached with 0.25% trypsin, washed with PBS, and permeabilized with 70% ethanol (10 minutes at 4° C.). For direct assays, cells are immunolabelled with the following antihuman antibodies: CD13-PE, CD31-FITC, CD54-PE, CD90-FITC, CD51/CD61-FITC (Pharmingen, Los Angeles, Calif., USA), CD14-PE, CD38-FITC, CD34-PE (Dako, Glostrup, Denmark), CD29-FITC, CD45-PerCP, CD49d-PE, CD49e-FITC, CD64-FITC (Becton-Dickinson, San Jose, Calif., USA) and/or CD106-FITC (R&D Systems, Abingdon, UK). As controls, mouse IgG₁-PE, IgG₁-FITC, IgG₁-perCP, or IgG₂ₙ-PE (Becton-Dickinson) are used. For indirect assays, cells are immunolabelled with the following anti-human antibodies: SH2, SH3, SH4 (Osiris Therapeutics, Baltimore, Md., USA), von Willebrand factor (Pharmingen), alpha-smooth muscle actin, ASMA (Sigma) or Mab1470 (Chemicon, Temecula, Calif., USA). As secondary antibodies, anti mouse IgGwm-FITC or -PE (Sigma) are used. Labelled cells are analysed either by epifluorescence microscopy or by flow cytometry. In the latter case, 10,000 events are acquired and analysed in a FACScan flow cytometer (Becton Dickinson) using CELLQUEST software.

### Example 3

### In Vitro Adipogenic Differentiation of UCB-Derived Pluripotent Mesenchymal Cells

Pluripotent cells are cultured in H15100 containing 10⁻⁶ M dexamethasone, 50 μg/mL ascorbic acid and 10 mM β-glycerolphosphate, resulting in partial differentiation of pluripotent cells towards adipocytes as demonstrated by Oil Red staining (Ramirez-Zacarias et al, Histochemistry 97: 493-7, 1992).

### Example 4

In Vitro Neurogenic Differentiation of UCB-Derived Pluripotent Mesenchymal Cells

Mononuclear cord blood cells obtained as described in Example 1 are cultured High Dulbecco's MEM (GibcoBRL) supplemented with 30% fetal calf serum (FCS) containing glutamine (0.02 mM) and penicillin/streptomycin (100 U/mL) in normal tissue culture-flasks (Nunclon). For differentiation, cells are seeded on glass cover slips coated with 1 mg/mL poly-D-lysine and 13 μg/mL laminin and incubated in a differentiation medium XXL containing Dulbecco's MEM, 15% heat inactivated FCS, 100 U/mL penicillin/streptomycin, 50 ng/nL nerve growth factor, 10 ng/mL bFGF, 1 mM dibutyryl camp, 0.5 mM IBMX, and 10 μM retinoic acid for at least 14 days.

After the induction period (27 days) cells are fixed according to a standard protocol (Rosenbaum et al., Neurobiol. Dis. 5: 55-64, 1998) and stained with antibodies against neural specific antigens. Specimen are analyzed using fluorescence and transmission light microscopy.

### Example 5

In Vitro Haematopoietic Differentiation of UCB-Derived Pluripotent Mesenchymal Cells

Pluripotent UCB cells are expanded for two weeks in the presence of a hematopoetic specific culture medium, with a growth factor mixture containing hu-Flt3-L (CellGenix), hu-SCF (CellGenix), IL-3 (Cellsystems), hu-IL-6 (Cellsystems), hu-TPO (CellGenix), and hu-G-CSF (Amgen). Human progenitor colony-forming assay on days 0 and 14 are performed by applying a ready-to-use methylcellulose medium (Methocult 4434, Stem Cell Technologies).

### Example 6

### In Vivo Hepatic Differentiation of UCB-Derived Pluripotent Mesenchymal Cells in Mice

Following the procedure of Beerheide et al., Biochem. Biophys. Research Comm. 294: 1052-63, 2002, SCID mice (age: 6-10 weeks, 18-22 g) are anesthesized by i.p. injection of 61.5 mg/kg ketamine and 2.3 mg/kg xylazine, which were combined immediately before administration. In one procedure, hepatectomy is performed on liver lobe number 1 (the large lobe directly under the right and left upper main liver lobes (lobes nos. 2 and 3) by ligating and excising it. A stem cell suspension (2×10⁵ human umbilical cord stem cells of the present invention suspended in 100 μL of William's E medium) is slowly injected into the subcapsular parenchyma of liver lobe no. 2 using a 26-gauge needle. In another procedure, hepatectomy is not performed and the stem cells are transplanted directly into liver lobe no. 1. The transdifferentiation of human UCB cells that are incorporated can be determined by performing immunohistochemistry on liver tissue of stem cell transplant recipients using a monoclonal antibody that cross-reacts with human albumin and not murine albumin.

### Example 7

In Vivo Hematopoietic Differentiation of UCB-Derived Pluripotent Mesenchymal Cells in Sheep

Following the procedure of Flake et al., Science 233: 776-8, 1986, 1500 UCB stem cells of the invention are injected intraperitoneally into preimmune fetal sheep. Eight months after the transplantation procedure, the transdifferentiation of human UCB cells into hematopoietic cells can be determined by examination of the cross-reactivity of heart specimens (atria, ventricles, and septum) from transplant recipients with anti-HSP27 monoclonal antibody, which is specific for human heat shock protein.

### Example 8

### In Vivo Hepatic Differentiation of UCB-Derived Pluripotent Mesenchymal Cells in Sheep

UCB stem cells of the invention are injected intraperitoneally into preimmune fetal sheep using the procedure used in Example 7 above. Fourteen months after the transplantation procedure, the transdifferentiation of human UCB cells into hepatic cells can be determined by examination of the cross-reactivity of liver specimens from transplant recipients using a monoclonal antibody that cross-reacts with human albumin but not with sheep albumin.

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating a vascular, muscle, hepatic, pancreatic, or neural disease, said method comprising the step of administering to a patient a pluripotent cell or a progeny cell derived therefrom prepared from human umbilical cord blood, placental blood, or a blood sample from a newborn human, wherein said pluripotent cell
(a) expresses SH2, SH3, SH4, CD13, CD29, CD49e, CD54, and CD90 antigen markers;
(b) does not express CD14, CD31, CD34, CD45, CD49d, or CD106 antigen markers; and
(c) is capable of differentiating into one or more of a mesenchymal pluripotent cell, a hematopoietic pluripotent cell, a neural pluripotent cell, or an endothelial pluripotent cell.

2. The method of claim 1, wherein said disease is a vascular disease.

3. The method of claim 1, wherein said disease is a smooth muscle or cardiac muscle disease.

4. The method of claim 1, wherein said disease is a hepatic disease.

5. The method of claim 1, wherein said disease is a pancreatic disease.

6. The method of claim 1, wherein said disease is a neural disease.

7. The method of claim 1, wherein said method comprises administering said cell to effect organ regeneration.

8. The method of claim 1, wherein multiple said cells are used to grow a blood vessel in vitro, which is implanted in said patient.

9. The method of claim 1, wherein said progeny cell of said pluripotent cell is administered to said patient.

10. The method of claim 9, further comprising inducing said progeny cell to express an endothelial cell marker before administering said progeny cell to said patient.

11. The method of claim 9, wherein said progeny cell expresses a marker recognized by a P1H12 monoclonal antibody.

12. The method of claim 9, further comprising inducing said progeny cell to express a liver cell marker before administering said progeny cell to said patient.

13. The method of claim 9, further comprising inducing said progeny cell to express a pancreatic cell marker before administering said progeny cell to said patient.

14. The method of claim 9, further comprising inducing said progeny cell to express a nerve cell marker before administering said progeny cell to said patient.

15. The method of claim 9, further comprising inducing said progeny cell to express a cardiac or smooth muscle cell marker before administering said progeny cell to said patient.

16. A method of whether a test agent induces differentiation of an isolated pluripotent cell, said method comprising contacting said pluripotent cell characterized by the expression of SH2, SH3, SH4, CD 13, CD29, CD49e, CD54, and CD90 antigens, and lacking the expression of CD14, CD34, CD45, CD49d, and CD106 antigens with said test agent and detecting a change in marker expression of said contacted pluripotent cell, wherein said change indicates that said test agent induces differentiation of said isolated pluripotent cell.

17. A method for producing a population of cells characterized by the expression of SH2, SH3, SH4, CD13, CD29, CD49e, CD54, and CD90 antigen markers, and lacking the expression of CD14, CD34, CD45, CD49d, and CD106 antigen markers, said method comprising the steps of:
a. providing pluripotent cells derived from umbilical cord blood and capable of differentiating into mesenchymal pluripotent cells, hematopoietic pluripotent cells, neural pluripotent cells, or endothelial pluripotent cells;
b. culturing said pluripotent cells in a medium containing dexamethasone for a time sufficient to expand said population of pluripotent cells; and
c. isolating said pluripotent cells from said culture, wherein greater than 20% of said isolated pluripotent cells are positive for SH2, SH3, SH4, CD13, CD29, CD49e, CD54, and CD90 markers, and negative for CD14, CD34, CD45, CD49d, and CD106 markers.

18. A composition comprising multiple pluripotent cells that are positive for SH2, SH3, SH4, CD13, CD29, CD49e, CD54, and CD90 markers, and negative for CD14, CD34, CD45, CD49d, and CD106 markers, suspended a pharmaceutically acceptable carrier.

19-32. (canceled)

33. The composition of claim 18, wherein the pharmaceutically acceptable carrier is selected from the group consisting of saline, a gel, a hydrogel, a sponge, and a matrix.

34. A pluripotent progeny cell obtained by the in vitro or ex vivo transfection with DNA encoding a desired protein of a pluripotent cell positive for SH2, SH3, SH4, CD13, CD29, CD49e, CD54, and CD90 markers, and negative for CD14, CD34, CD45, CD49d, and CD106 markers.

35. A composition comprising multiple cells of claim 34, suspended in a pharmaceutically acceptable carrier.

36. The composition of claim 34, wherein the pharmaceutically acceptable carrier is selected from the group consisting of saline, a gel, a hydrogel, a sponge, and a matrix.

37. A therapeutic method comprising administering to a patient in need thereof a therapeutically effective amount of the composition of claim 35.

38. The method of claim 36, wherein said cells express in said patient a therapeutically effective amount of said desired protein.

39. The method of claim 34, wherein said pluripotent cell is capable of differentiating into all of the same cell types as said pluripotent cells.

40. Use in the preparation of a medicament for the treatment of a vascular, hepatic, pancreatic or neural disease, of a pluripotent cell or progeny thereof prepared from umbilical cord blood, placental blood, or blood prepared from a newborn human, wherein said pluripotent cell a) is positive for SH2, SH3, SH4, CD13, CD29, CD49e, CD54, and CD90 markers, b) is negative for CD14, CD34, CD45, CD49d, and CD106 markers, and c) is capable of differentiating into one or more of a mesenchymal pluripotent cell, a hematopoietic pluripotent cell, a neural pluripotent cell, or an endothelial pluripotent cell.

41. The use of claim 40, wherein said disease is a vascular disease.

42. The use of claim 40, wherein said disease is a smooth muscle or cardiac muscle disease.

43. The use of claim 40, wherein said disease is a hepatic disease.

44. The use of claim 40, wherein said disease is a pancreatic disease.

45. The use of claim 40, wherein said disease is a neural disease.

46. The use of claim 40, wherein the cells are used for organic regeneration.

47. The use of claim 40, wherein said cell is a said progeny cell that expresses the endothelial cell marker P1H12.
